(19) United States
(12) Patent Application Publication
(10) Pub. No.: US 2020/0080014 A1
(43) Pub. Date: Mar. 12, 2020

(54) GTBE COMPOSITIONS, METHODS AND INSTALLATIONS FOR ENHANCED OCTANE BOOSTING

(71) Applicant: PROCEDE HOLDING B.V.

(72) Inventors: Geert Frederik VERSTEEG, Enschede (NL); Wouter Nicolaas WERMINK, Enschede (NL)

(73) Assignee: PROCEDE HOLDING B.V., Enschede (NL)

(21) Appl. No.: 16/063,586

(22) PCT Filed: Dec. 19, 2016
(86) PCT No.: PCT/NL2016/050889

§ 371 Date: Jun. 18, 2018

(30) Foreign Application Priority Data
Dec. 18, 2015 (CA) 2915852

(51) Int. Cl. C10L 1/185; C10L 10/10; C07C 41/06

(52) U.S. Cl. CPC C10L 1/1852; C10L 10/10; C10L 2270/023; C10L 2200/0423; C10L 2200/0476; C07C 41/06

(57) ABSTRACT

Techniques for boosting octane of gasoline can include using glycerol and isobutene in a reaction vessel under conditions to generate a blend of Glycerol Tert-Butyl Ethers (GTBE) comprising mono-GTBEs, di-GTBEs and tri-GTBE; regulating the conditions to favor production of mono-GTBEs according to equilibrium reactions, thereby producing a mono-shifted GTBE blend; and using the mono-shifted GTBE blend with gasoline as an octane booster. If a GTBE blend with further increased octane boosting capacity is desired, a purification/separation step can be included in the process to increase the mono-GTBE content of the mono-shifted GTBE blend. For blends with target density or viscosity, the production can be regulated to obtain a GTBE blend with an increase or decrease in density and/or viscosity. Compositions, processes, methods and installations are described for providing enhancements in the production and use of GTBE and its components.

### TECHNICAL FIELD

The technical field generally relates to octane boosting for gasoline.

### BACKGROUND

There are various compounds that have been used for octane boosting. For example, blends of Glycerol Tert-Butyl Ethers (GTBE) have been used to boost octane for gasoline. These GTBE blends have consisted of mixtures of mono-, di- and tri-GTBE and have been able to achieve limited octane booster/enhancer ability.

### SUMMARY

In some implementations, there is a method of boosting octane of gasoline, comprising: contacting glycerol and isobutene in a reaction vessel under conditions to generate a blend of Glycerol Tert-Butyl Ethers (GTBE) comprising mono-GTBEs, di-GTBEs and tri-GTBE; regulating the conditions to favor production of mono-GTBEs according to equilibrium reactions, thereby producing a mono-shifted GTBE blend; and using the mono-shifted GTBE blend with gasoline as an octane booster.

In some implementations, the step of regulating comprises controlling molar ratios of the glycerol and the isobutene. In some implementations, the controlling the molar ratio of isobutene/glycerol is done between 1/4 and 4/1. In some implementations, the molar ratio of isobutene/glycerol is between 1/3 and 3/1. In some implementations, the molar ratio of isobutene/glycerol is between 1/2.5 and 2.5/1. In some implementations, the molar ratio of isobutene/glycerol is between 1/2 and 2/1. In some implementations, the controlling the molar ratio of isobutene/glycerol is done between 1/1 and 4/1. In some implementations, the molar ratio of isobutene/glycerol is between 1.5/and 2.5/1. In some implementations, the molar ratio of isobutene/glycerol is between 1.75/and 2.25/1. In some implementations, the molar ratio of isobutene/glycerol is between 1.9/and 2.1/1.

The mono-shifted GTBE blend can be optionally increased in mono-GTBE content by the use of a suitable separation step.

In some implementations, the molar ratio of isobutene/glycerol is regulated to maximize production of mono-GTBEs relative to di-GTBEs and tri-GIBE.

In some implementations, the molar ratio of isobutene/glycerol, reaction temperature, and residence time are provided to obtain a yield of the mono-GIBE of at least 10%. In some implementations, the molar ratio of isobutene/glycerol, reaction temperature, and residence time are provided to obtain a yield of the mono-GIBE of at least 15%. In some implementations, the molar ratio of isobutene/glycerol, reaction temperature, and residence time are provided to obtain a yield of the mono-GIBE of at least 20%. In some implementations, the molar ratio of isobutene/glycerol, reaction temperature, and residence time are provided to obtain a yield of the mono-GIBE of at least 25%. In some implementations, the molar ratio of isobutene/glycerol, reaction temperature, and residence time are provided to obtain a yield of the mono-GIBE of at least 30%. In some implementations, the molar ratio of isobutene/glycerol, reaction temperature, and residence time are provided to obtain a yield of the mono-GIBE of at least 35%. In some implementations, the molar ratio of isobutene/glycerol, reaction temperature, and residence time are provided to obtain a yield of the mono-GIBE of at least 40%. In some implementations, the molar ratio of isobutene/glycerol, reaction temperature, and residence time are provided to obtain a yield of the mono-GIBE of at least 45%. In some implementations, the molar ratio of isobutene/glycerol, reaction temperature, and residence time are provided to obtain a yield of the mono-GIBE between 20% and 50% or between 20% and 35%

In some implementations, the reaction temperature is provided between 40° C. and 100° C. In some implementations, the reaction temperature is provided between 45° C. and 95° C. In some implementations, the reaction temperature is provided between 50° C. and 90° C. In some implementations, the reaction temperature is provided between 55° C. and 85° C. In some implementations, the reaction temperature is provided between 40° C. and 85° C. In some implementations, the reaction temperature is provided between 45° C. and 80° C. In some implementations, the reaction temperature is provided between 50° C. and 75° C. In some implementations, the reaction temperature is provided between 55° C. and 70° C.

In some implementations, the mono-GIBE content of the GIBE exiting the reactor can be increased by the use of a suitable separation step, e.g. via extraction or (vacuum) distillation.

In some implementations, and depending on the regulation of the reactor settings for the production of the GTBEs, the concentration of the mono-GTBEs in the mono-GIBE, di-GTBE and tri-GIBE mixture is increased to 15% by the use of a suitable separation step. In some implementations, and depending on the regulation of the reactor settings for the production of the GTBEs, the concentration of the mono-GTBEs in the mono-GTBE, di-GIBE and tri-GIBE mixture is increased to 20% by the use of a suitable separation step. In some implementations, and depending on the regulation of the reactor settings for the production of the GTBEs, the concentration of the mono-GTBEs in the mono-GIBE, di-GIBE and tri-GIBE mixture is increased to 40% by the use of a suitable separation step. In some implementations, and depending on the regulation of the reactor settings for the production of the GTBEs, the concentration of the mono-GTBEs in the mono-GIBE, di-GIBE and tri-GIBE mixture is increased to 60% by the use of a suitable separation step. In some implementations, and depending on the regulation of the reactor settings for the production of the GTBEs, the concentration of the mono-GTBEs in the mono-GIBE, di-GIBE and tri-GIBE mixture is increased to 80% by the use of a suitable separation step. In some implementations, and depending on the regulation of the reactor settings for the production of the GTBEs, the concentration of the mono-GTBEs in the mono-GIBE, di-GIBE and tri-GIBE mixture is increased between 20% to 100% by the use of a suitable separation step.

In some implementations, there is provided an octane boosting composition comprising Glycerol Tert-Butyl Ethers (GIBE) consisting of 3-tert-Butoxypropane-1,2-diol and 2-tert-Butoxypropane-1,3-diol.

In some implementations, there is provided an octane boosting composition consisting of mono-GTBEs, di-GTBEs and tri-GIBE, wherein the composition is formulated such that a concentration of the mono-GTBEs therein is sufficient to increase the octane number by at least 0.1 compared to a composition consisting of di-GTBEs and tri-GIBE.

In some implementations, the concentration of the mono-GTBEs is sufficient to increase the octane number by at least 1.0 compared to the composition consisting of di-GTBEs and tri-GIBE. In some implementations, the concentration of the mono-GTBEs is sufficient to increase the octane number by at least 2.0 compared to the composition consisting of di-GTBEs and tri-GIBE. In some implementations, the concentration of the mono-GTBEs is sufficient to increase the octane number by at least 3.0 compared to the composition consisting of di-GTBEs and tri-GIBE. In some implementations, the concentration of the mono-GTBEs is sufficient to increase the octane number by at least 4.0 compared to the composition consisting of di-GTBEs and tri-GIBE. In some implementations, the concentration of the mono-GTBEs is sufficient to increase the octane number by at least 5.0 compared to the composition consisting of di-GTBEs and tri-GIBE. In some implementations, the concentration of the mono-GTBEs is sufficient to increase the octane number between 0.1 and 7.5 compared to the composition consisting of di-GTBEs and tri-GIBE. In some implementations, the concentration of the mono-GTBEs is sufficient to increase the octane number by at least 0.15 or at least 0.2 compared to the composition consisting of di-GTBEs and tri-GIBE.

In some implementations, there is provided an octane boosting composition comprising mono-GTBEs, di-GTBEs and tri-GIBE wherein a concentration of the mono-GTBEs is above 10 wt % on a total mass basis of all GTBEs.

In some implementations, the concentration of the mono-GTBEs is above 15 wt %, 20 wt %, 25 wt %, 30 wt %, 40 wt %, 60 wt %, or 80 wt %.

In some implementations, the composition further includes trimethylpentane, and may also include other compounds.

In some implementations, there is provided a method of producing Glycerol Tert-Butyl Ethers (GIBE) blends for use in different fuels, comprising: reacting glycerol and isobutene to produce a GIBE blend comprising mono-GTBEs, di-GTBEs and tri-GIBE according to equilibrium reactions; selectively shifting the equilibrium reactions to favour production of a predetermined GIBE blend, comprising: shifting the equilibrium reactions to favour production mono-GTBEs over di-GTBEs and tri-GIBE, to produce a mono-shifted GIBE blend suitable for use in as an octane booster, and shifting the equilibrium reactions to favour production of di-GTBEs and/or tri-GIBE, to produce a di-shifted and/or tri-shifted GIBE blend suitable for use in biodiesel.

Subsequently, the GIBE blend can be optionally increased in mono-GIBE, and/or di-GTBE and/or tri-GIBE content, by the use of a suitable separation step.

In some implementations, the shifting is performed by changing molar ratios of glycerol and isobutene reactants, changing residence time of the glycerol and isobutene, and/or changing a reaction temperature.

In some implementations, shifting the equilibrium reactions to produce the mono-shifted GIBE blend comprises at least one of the steps recited above or herein.

In some implementations, the method further includes separating the mono-shifted GIBE blend in order to increase mono-GIBE content and/or decrease content of di-GTBEs, tri-GIBE, and/or one or more other compounds. The separation can include vacuum based methods and/or extraction based methods.

In some implementations, there is provided an installation for production of multiple Glycerol Tert-Butyl Ethers (GIBE) blends, comprising: a reaction vessel comprising: a reaction chamber for accommodating the production of GIBE, at least one reactant inlet in fluid communication with the reaction chamber for supplying glycerol and isobutene therein, and a product outlet in fluid communication with the reaction chamber for withdrawing the GIBE blend; and a regulator configured for regulating conditions of the reaction vessel in alternating fashion between a first mode and a second mode, the first mode causing production of a mono-shifted GIBE blend and the second mode causing production of a di- and/or tri-shifted GIBE blend.

Optionally, there can be a GIBE separation unit to optionally increase the mono-GIBE content, or the di-GIBE and/or the tri-GIBE content. The GIBE separation unit can include a distillation unit (e.g., vacuum distillation unit), an extraction unit, and/or another type of separation unit.

In some implementations, the mono-shifted GIBE blend is suitable for use as an octane booster.

In some implementations, the di- and/or tri-shifted GIBE blend is suitable for use in biodiesel.

In some implementations, the regulator is configured to modify molar ratios of the glycerol and the isobutene supplied to the reaction chamber.

In some implementations, the regulator is coupled to the at least one reactant inlet to regulate the molar ratios.

In some implementations, the regulator is configured to modify a reaction temperature in the reaction chamber.

In some implementations, the regulator is configured to modify a residence time of reactants in the reaction chamber.

In some implementations, the regulator is coupled to the product outlet to regulate withdrawal of the GIBE blend.

In some implementations, the regulator is further configured for regulating conditions of the reaction vessel in a third mode.

In some implementations, the installation includes a first storage vessel and a second storage vessel coupled to the product outlet of the reaction vessel, and flow direction system adapted to control flow of the GIBE blend into the first or second storage vessel respectively in the first and second modes.

In some implementations, the regulator is configured to enable one or more of the steps and/or obtain features as recited above and/or herein.

In some implementations, the content of the mono-shifted GIBE blend, or the content of the di-shifted GIBE and/or tri-shifted GIBE blend can be further increased by the use of a suitable separation step, e.g., via extraction or vacuum distillation.

In some implementations, the content of mono-GIBE, di-GIBE or tri-GIBE can be shifted in such a manner that a GIBE blend with specific density and/or viscosity is obtained. The viscosity and/or density properties of the GTBEs can be leveraged in various ways in order to enhance mixing, GIBE blend production and performance. Production of GIBE blends can be tailored to a particular fuel to which it is added in order to have viscosity, density, octane-boosting and/or other properties of the blend that have an impact on the fuel's properties (e.g., boosting of octane and increase in density with a mono-shifted blend). Properties such as viscosity and density can be provided so that the GIBE blend has enhanced producibility, post-production mixing with other additives or the fuel, and/or effects on the fuel after mixing therewith, which may be in addition to other advantageous effects such as octane-boosting (e.g., for gasoline) and other effects (e.g., for biodiesel). The composition of the GIBE blend can be formulated and/or generated via equilibrium shifting/control, so that the end product has multiple functionalities that are balanced for particular applications.

### BRIEF DESCRIPTION OF DRAWINGS

FIG. 1 is an image of the production of GIBE products from glycerol and isobutene.

FIG. 2 is a table illustrating test data regarding octane boosting of GIBE products.

FIG. 3 is an image of the side-product spectrum in the synthesis of GIBE® from glycerol and isobutene.

FIG. 4 is a schematic scheme of a GIBE production set-up.

FIGS. 5 and 6 are tables showing experimental results.

FIG. 7 is another schematic scheme of a GIBE production set-up.

FIG. 8 is a graph of Conversion of IB for experiments performed with varying molar ratio of IB: glycerol and at varying temperatures. The source of isobutene was 50 vol %/50 vol % IB/1-butene.

FIG. 9 is a graph of Conversion of glycerol for experiments performed with varying molar ratio of IB: glycerol and at varying temperatures. The source of isobutene was 50 vol %/50 vol % IB/1-butene.

FIG. 10 is a graph of Yield of mono-GIBE for experiments performed with varying molar ratio of IB: glycerol and at varying temperatures. The source of isobutene was 50 vol %/50 vol % IB/1-butene.

FIG. 11 is a graph of Yield of di-GIBE for experiments performed with varying molar ratio of IB: glycerol and at varying temperatures. The source of isobutene was 50 vol %/50 vol % IB/1-butene.

FIG. 12 is a graph of Yield of tri-GIBE for experiments performed with varying molar ratio of IB: glycerol and at varying temperatures. The source of isobutene was 50 vol %/50 vol % IB/1-butene.

FIG. 13 is a schematic of an experimental setup used for kinetic and equilibria investigations of a GIBE system.

FIG. 14 is a schematic scheme of an experimental setup used for equilibria investigations of a GIBE system.

FIG. 15 is a graph of the concentration profile obtained for the reaction of pure mono-GTBE with H₂SO₄ at a temperature of 50° C.

FIG. 16 is a table showing the experimental results of the EQ compositions of pure mono-GIBE with sulphuric acid at varying temperatures.

FIG. 17 is a table showing the experimental results of the EQ compositions of pure di-GTBE with sulphuric acid at varying temperatures.

FIG. 18 is a graph showing the equilibrium conversions of isobutene and glycerol in the production of GIBE from pure isobutene and pure glycerol as a function of reactant ratio at a temperature of 50° C.

FIG. 19 is a graph showing the equilibrium conversions of isobutene and glycerol in the production of GIBE from pure isobutene and pure glycerol as a function of reactant ratio at a temperature of 80° C.

FIG. 20 is a graph showing the equilibrium conversions of tert-butyl alcohol and glycerol in the production of GIBE from pure tert-butyl alcohol and pure glycerol as a function of reactant ratio at a temperature of 80° C.

FIG. 21 is a graph showing selectivities of isobutene to products in the production of GIBE from pure isobutene and pure glycerol as a function of reactant ratio at a temperature of 50° C.

FIG. 22 is a graph showing selectivities of isobutene to products in the production of GIBE from pure isobutene and pure glycerol as a function of reactant ratio at a temperature of 80° C.

FIG. 23 is a table illustrating test data regarding octane boosting of pure mono-GIBE and pure di-GIBE at varying concentrations.

FIG. 24 is a graph of PT-curves of pure mono-GIBE, pure di-GIBE and pure tri-GIBE

FIG. 25 is a table showing compositions of the bottom sample and top fractions of a mono-GIBE vacuum distillation run.

FIG. 26 is a table showing compositions of the bottom sample and top fractions of a di-GTBE vacuum distillation run.

FIG. 27 is a table showing compositions of the bottom sample and top fractions of a tri-GTBE vacuum distillation run.

FIG. 28 is a graph showing the densities of mono-GIBE, di-GIBE and tri-GIBE as a function of temperature.

FIG. 29 is a graph showing the dynamic viscosities of mono-GIBE, di-GIBE and tri-GTBE as a function of temperature.

### DETAILED DESCRIPTION

In some implementations, a GIBE-based octane booster composition can be produced to have increased mono-GIBE content, and such mono-shifted GIBE blend can be useful for octane boosting particularly due to the enhanced octane boosting effects of mono-GIBE compared to di- and tri-GIBE. Methods and systems for producing a mono-shifted GIBE blend are also provided. In addition, an installation can be provided for selectively producing different GIBE blends for different uses.

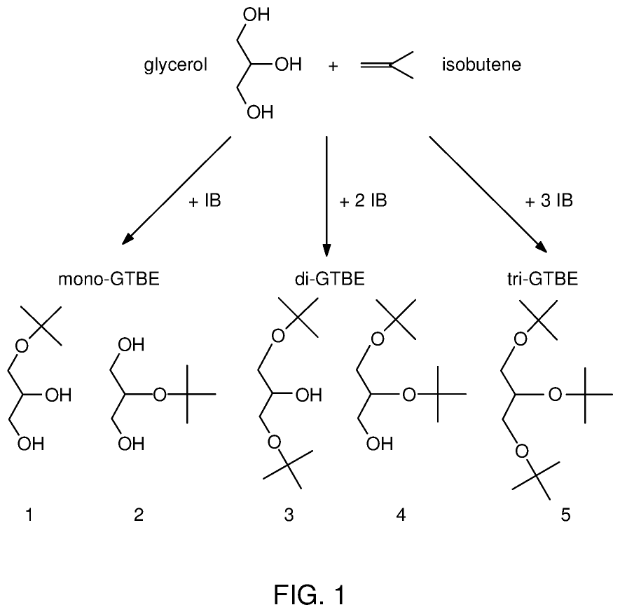

GIBE is a mixture of tert-butyl ethers of glycerol and there are five possible GIBE molecules: two isomers of mono-GIBE; two isomers of di-GIBE, and tri-GIBE. The production of GIBE includes several equilibrium reaction steps, shown in FIG. 1. Glycerol and isobutene are used as reactants. A homogeneous or heterogeneous catalyst can be used to enhance the reaction rate.

The IUPAC names of the products illustrated in FIG. 1 are (1) 3-tert-Butoxypropane-1,2-diol; (2) 2-tert-Butoxypropane-1,3-diol; (3) 1,3-di-tert-Buoxypropane-2-ol; (4) 2,3-di-tert-Butoxypropane-1-ol; and (5) 1,2,3-di-tert-Butoxyropane. Products (1) and (2) will be referred to herein as mono-GTBE, products (3) and (4) will be referred to herein as di-GTBE and product (5) will be referred to herein as tri-GTBE.

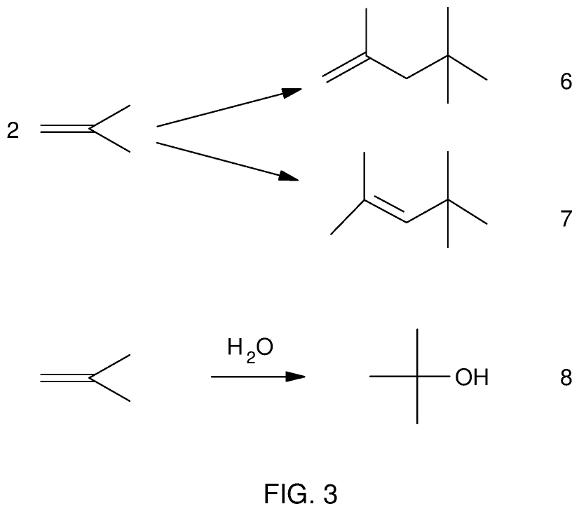

It should also be noted that undesired oligomerization and hydration of isobutene can occur. Such reactions are shown in FIG. 3. The IUPAC names for the side-products are (6) 2,4,4-Trimethyl-1-pentene; (7) 2,4,4-Trimethyl-2-pentene; and (8) 2-Methyl-2-propanol (or tert-butanol). Products (6) and (7) will be referred to as TMP and product (8) will be referred to as TBA.

It is also noted that tert-butyl alcohol can react with glycerol to form GTBE. In addition, gas streams containing isobutene (e.g., raffinate-I) can be used as a reactant. Compounds can react to form isobutene for the production of GTBE (e.g., tert-butyl alcohol or isobutanol dehydration).

In some implementations, glycerol and isobutene are contacted in a reaction vessel under conditions to generate a blend of Glycerol Tert-Butyl Ethers (GTBE) comprising mono-GTBEs, di-GTBEs and tri-GTBE. The reaction vessel conditions and the molar ratios of the glycerol and isobutene reactants can be regulated to favor production of mono-GTBEs according to equilibrium reactions, thereby producing a mono-shifted GTBE blend. The mono-shifted GTBE blend can be used with gasoline as an octane booster, and due to the higher mono-GTBE content the octane boosting is enhanced.

Referring to FIG. 1, the amount of isobutene required to produce GTBE is highest for tri-GTBE. As glycerol is the cheapest reactant, and isobutene is the most expensive reactant, it is additionally advantageous to produce and use mono-GTBE as a product because production costs can be reduced compared to the counterpart di- and tri-GTBE. Glycerol can be obtained economically due to overproduction in the biodiesel industry, for example. Thus, mono-shifted GTBE blends can not only increase octane boosting effects but can also be produced more economically and using less isobutene reactant.

The GTBE production reactions are equilibrium reactions. Investigations were conducted to assess manipulation of the process to shift production toward certain types of GTBE (e.g., transform di-GTBEs into mono-GTBEs or impact proportion of products based on molar ratios of reactants entering the reactor vessel), and to assess to what extent pure GIBE compounds can be converted to other GIBE compounds. Studies revealed that it is possible to manipulate the GIBE type during production, and shift the proportions of the GTBEs being produced and convert pure GIBE compounds to other GIBE compounds. Moreover, varying the molar ratio of the reactants influences reactor composition. For example, if mono-GIBE is desired in a specific time, the process can be manipulated to shift toward increased production of mono-GTBEs. Likewise, if a greater proportion of di-GIBE is desired, the process can be manipulated to optimize that type of GIBE being produced. Based on the conducted experimental work a thermodynamic model was constructed to predict equilibrium compositions at varying settings, e.g., varying temperatures and varying reactant ratios. Subsequent to the production step, a separation step could be included to further purify mono-GIBE, or di-GTBE, or tri-GIBE, for example by extraction or (vacuum) distillation.

Experiments were conducted to investigate the influence of temperature, isobutene to glycerol molar ratio, and the source of isobutene on the production of GIBE. Additional experiments were performed to assess the extent to which pure GIBE compounds can be converted to other GIBE compounds. With respect to purification, studies were performed to determine the PT-curves of the varying pure GTBEs, which were obtained after purification via vacuum distillation.

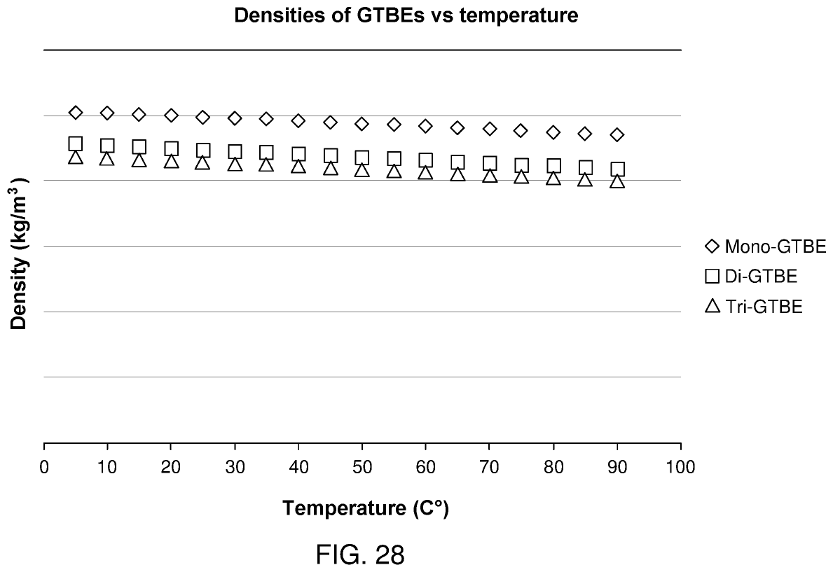

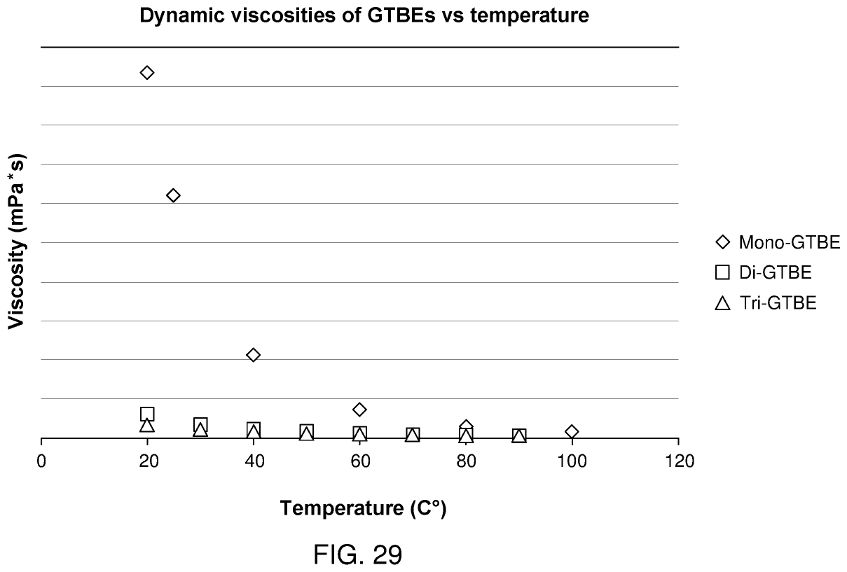

In some implementations, GIBE blend with specific characteristics with respect to density and dynamic viscosity could be desirable. Experiments were conducted to determine the difference in density and dynamic viscosity among the varying types of GIBE, and determine the effect of temperature on both the densities and dynamic viscosities of the different GTBEs (i.e. pure mono-GIBE, pure di-GIBE, and pure tri-GTBE). Results are shown in Experimentation 9 below and FIGS. 28 and 29.

Manipulating viscosity an/or density properties can be performed in concert with the particular composition of the GIBE blend and its end use (e.g., in gasoline). Such manipulations can be done during the production of GIBE blend to obtain a blend having target viscosity and/or density properties, or after the GIBE blend is formed in the context of mixing the GIBE blend with an end-use product. For example, when a GIBE blend having a target viscosity and/or density properties (e.g., target ranges of viscosity and/or density) is desired, the process conditions can be regulated to obtain certain amounts or proportions of mono-, di-, and tri-GTBE such that the aggregate properties are within the target range. For instance, if a GIBE blend with as much mono-GTBE as possible while staying below a target threshold viscosity value is desired, the process conditions can be operated to shift the reaction equilibria to produce the GIBE blend having a certain amount of mono-GIBE which have high viscosity and would thus increase the aggregate viscosity of the blend. If particular density or viscosity profiles are desired for the GIBE blend, the process conditions can be regulated to shift the reaction equilibria in order to produce the GIBE blend with the target properties.

Another example of leveraging GIBE viscosity manipulation is by increasing the temperature of the GIBE blend based on its composition (e.g., based on its mono-GIBE content which causes higher viscosity) for enhanced mixing with an end-product hydrocarbon, such as gasoline or biodiesel. Mixing of liquids (e.g., GTBE blend and gasoline) can be enhanced when the two liquids have similar viscosities. For a given GTBE blend with a high mono-GTBE content, the mixing temperature of the GTBE blend can be increased in accordance with the high mono-GTBE content to reduce the viscosity down below a target level (e.g., similar to that of the liquid into which the GTBE will be mixed). FIG. 29 shows that mono-GTBE has high viscosity at temperatures below 30° C. or 20° C., but as the temperature is increased the viscosity dramatically decreases. Thus, for high mono-GTBE blends, higher mixing temperatures can provide enhanced production of the octane boosted fuel.

Yet another way to leverage GTBE viscosity manipulation is in the context of adding a particular GTBE compound (e.g., mono-GTBE) to a GTBE blend in order to increase the amount of that GTBE compound in the blend. By controlling the temperature of the GTBE blend and/or the added GTBE compound, similar viscosities can be achieved between the two liquids for enhanced mixing. For example, if mono-GTBE is to be added to a blend, then it can be pre-heated to a temperature at which the mono-GTBE blend has a similar or identical viscosity to the GTBE blend or is within a certain percentage (e.g., 50%, 40%, 30%, 20%, or 10%). For instance, the mono-GTBE can be preheated to at least 50° C., 60° C., 70° C., 80° C. or 90° C. so as titecrease its viscosity during mixing with a blend. When the blend has a certain mono-GTBE content, the blend can also be preheated to decreases its viscosity. In scenarios where the blend has a high mono-GTBE content and mono-GTBE is being added to it to further increase the mono-GTBE concentration, both the blend and the added mono-GTBE can be heated to temperatures (which may be the same or different) to provide similar or generally identical viscosities for mixing.

Furthermore, in some implementations, the viscosity and/or density properties of the GIBE blend can be tailored or predetermined such that the GIBE blend has properties that will have a desired impact on the fuel into which it is added. For example, the production process can be performed under conditions to shift the equilibrium reactions to produce a GIBE blend that has a certain composition of mono-, di- and tri-GIBE with higher mono-GIBE content and corresponding higher density characteristics so that the GIBE blend can increase the density of the fuel to which it is added. Thus, the GIBE blend can be provided with viscosity and/or density properties that will increase or decrease the viscosity and/or density of the fuel (e.g., gasoline, biodiesel, etc.) to which it is added, so that the GIBE blend can have multiple functionalities (e.g., octane booster and density booster).

It is also noted that mixing can be provided in the reaction vessel in which GIBE blends are produced, particularly when raffinate-I is the source of isobutene as two liquid phases will remain in the reactor throughout the GIBE production run.

### Experimentation 1

The influence of the molar ratio of isobutene to glycerol, as well as the temperature, were investigated on the production of GIBE. Pure isobutene and pure glycerol were used as reactants.

### Description of Setup

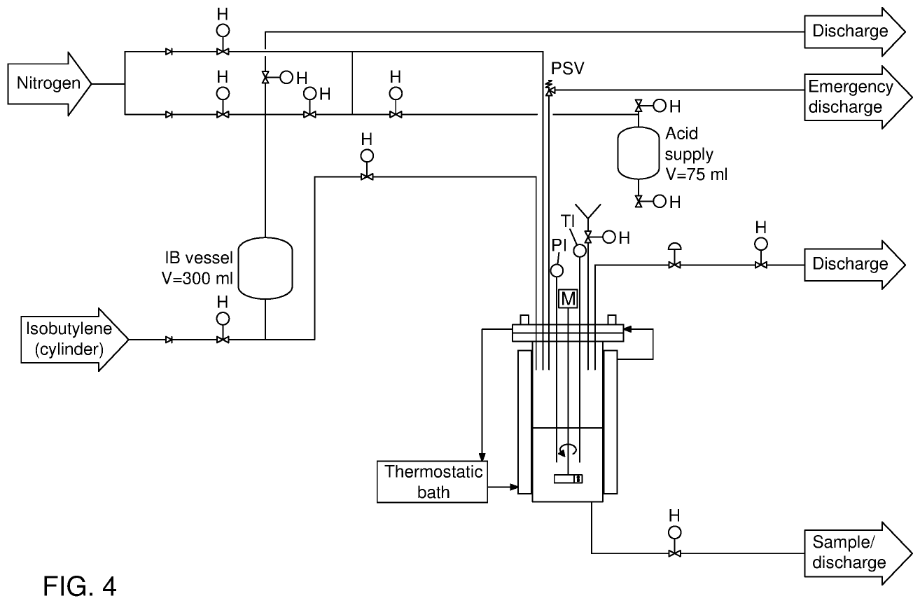

The GIBE setup included a batch wise operated stirred tank reactor with a total volume of 8 litres. The reactor was equipped with baffles, a jacket, a pressure gauge, a temperature indicator, a drain valve, a funnel, an isobutene dosing system, an acid dosing system and a pressure relief valve. The isobutene dosing system included an isobutene gas bottle, a 300 ml gas bomb and interconnecting tubing with manual operated valves. The reactor was heated with aid of a thermostatic bath with temperature control. The stirring speed could be manipulated with aid of a frequency converter. Each experiment continued until a significant drop in pressure was notified (several bars). The sulphuric acid content was 3 wt % of glycerol content. At the end of each experiment a liquid sample was taken for analysis. FIG. 4 shows a schematic scheme of the GIBE set-up.

### Results

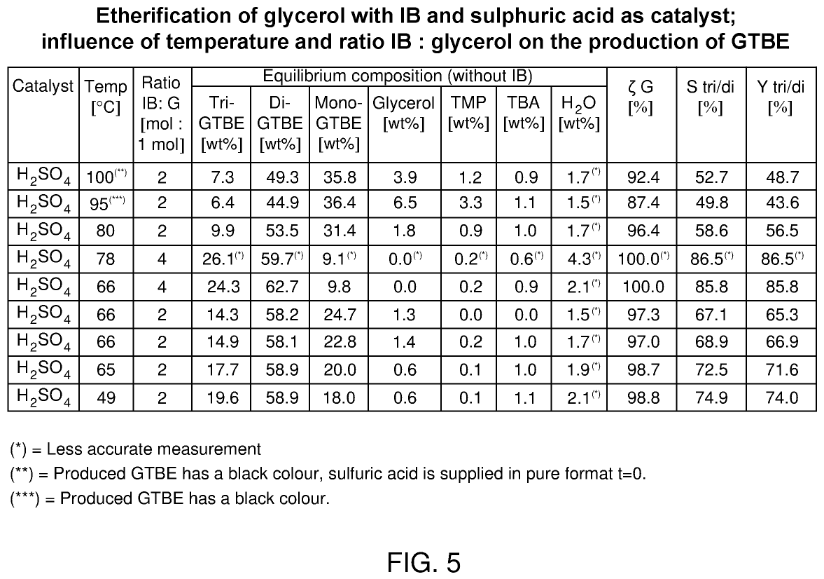

Table 1 illustrated as FIG. 5 shows the results of several IB to GIBE production experiments. From Table 1 the following conclusions were drawn:
(i) Above a reaction temperature of 80° C. the undesired formation of the by-product TMP increases to above 1 wt %.
(ii) Increasing the molar ratio of IB to glycerol from 2:1 to 4:1 the content of tri-GTBE increases 10-15 wt %, the content of di-GIBE increases 4-6 wt % and the content of mono-GIBE decreases about 15-22 wt %. In general it can be stated that the increase in molar ratio of IB to glycerol increases the yield of tri-GTBE and lowers the yield of mono-GIBE. Also the conversion of IB is lowered (not shown).
(iii) The glycerol conversion equals 97-100% for a molar ratio of IB to glycerol of 2:1 at a reaction temperature of 65° C. The tri- and di-GTBE selectivity respectively yield 67-72% and 65-72%. Increasing the temperature from 49° C. to 100° C. decreases the selectivity towards di- and tri-ethers for a molar ratio of IB to glycerol of 2:1.

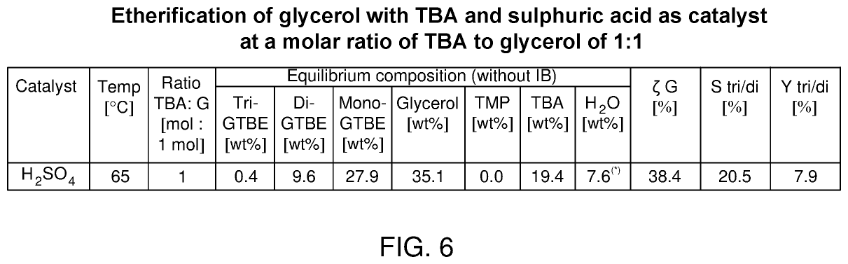

Table 2 illustrated as FIG. 6 shows the results of a TBA to GIBE production experiment. From Table 2 it can be concluded that performing the etherification with TBA instead of IB lowers the glycerol conversion and selectivity and yield of di- and tri-GIBE.

### Experimentation 2

The influence of the molar ratio of isobutene to glycerol, as well as the temperature, were investigated on the production of GIBE. A mixture of 50 vol %/50 vol % isobutene/1-butene and pure glycerol were used as reactants.

### Description of Setup

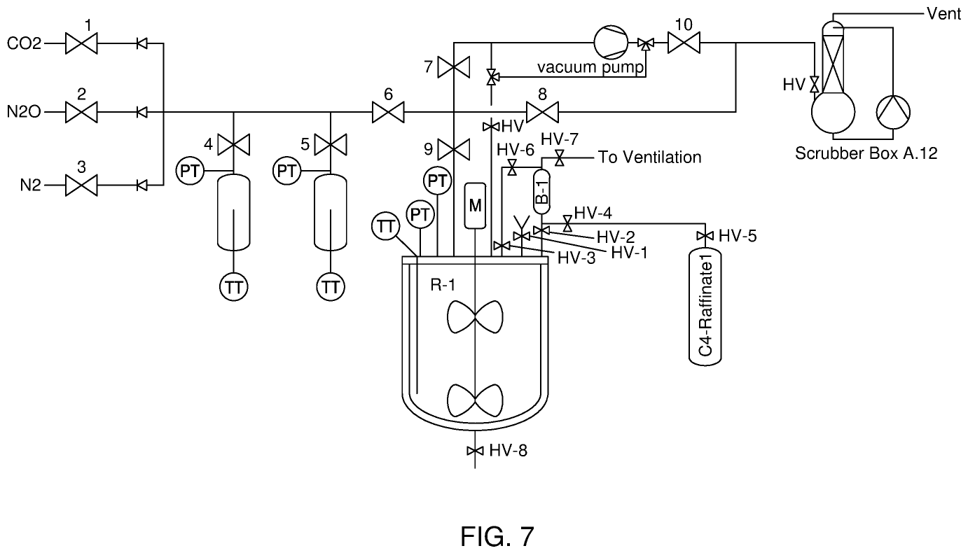

Experiments were performed in a 1 litre stainless steel autoclave. The autoclave could handle pressures up to 60 bar and temperatures up to 250° C. The reactor was equipped with glass windows. The reactor was operated batchwise. A vacuum pump was used to evacuate air in the reactor. Nitrogen could be fed to the reactor. The reactor temperature was regulated with a Julabo heater. A blade stirrer was operated with a magnetic drive. The blades were bent to enhance mixing of the two liquid phases and ensure sufficient interfacial area. Stirring speeds up to 1800 rpm could be achieved. Baffles inside the reactor promoted mixing. The temperature and pressure were monitored during each experiment. A gas bomb of 100 ml was used to enter isobutene to the reactor. Sampling was performed via a sampling valve at the bottom of the reactor. Samples were analysed by gas chromatography. FIG. 7 shows a schematic representation of the experimental setup.

### Results

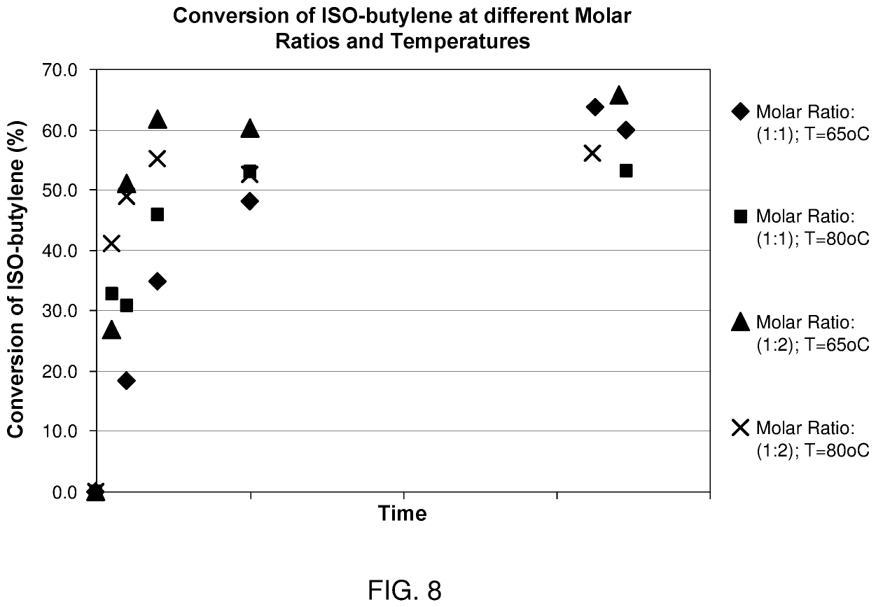

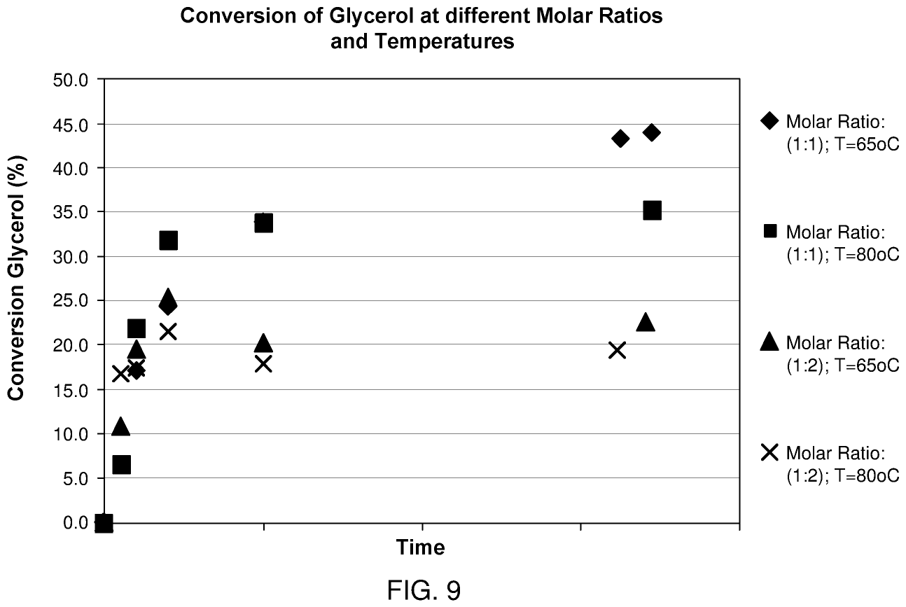

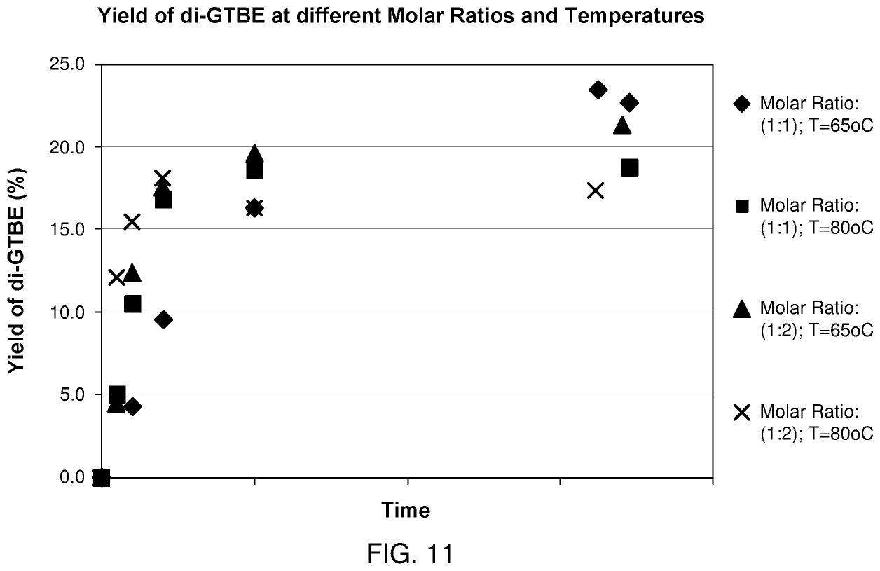

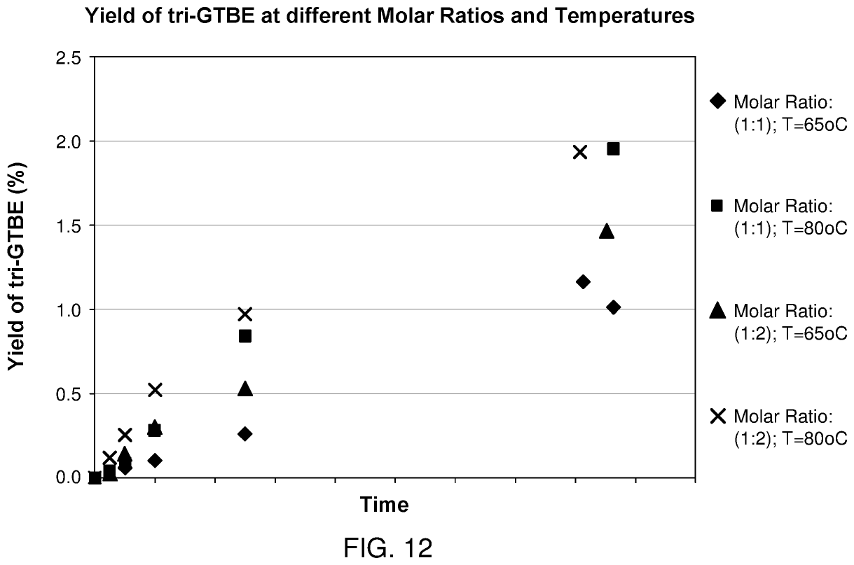

FIGS. 8 to 12 show the results of several 50 vol %/50 vol % isobutene/1-butene to GTBE production experiments.

For experiments performed with pure glycerol, conversions of glycerol were in the range of 20% to 45%. The highest glycerol conversion of 45% was accomplished with the experiment with an isobutene to glycerol molar ratio of 1:1 at 65° C. Lower conversions of glycerol were obtained at a molar ratio of 1:2. The isobutene conversion profiles follow the same trend as the total GTBE yield profiles. For experiments performed with pure glycerol, the conversion of isobutene is in the range of 55% to 65%. The highest yield of GTBE, i.e. mono-, di- and tri-GTBE, was observed for the experiment performed at 65° C. with an isobutene to glycerol molar ratio of 1:2.

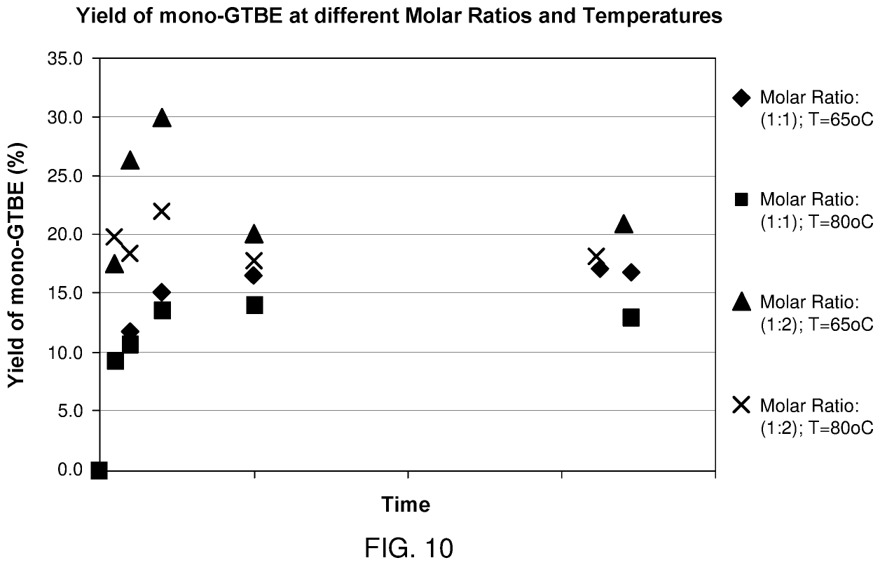

Interestingly, the mono-GTBE yield can be influenced via the residence time in the reactor, as depicted by FIG. 10. Therefore, when a high yield of mono-GTBE is desired, both the molar ratio of reactants, as well as the temperature and residence time in the reactor, can be used to steer the production of mono-GTBE. For example, in some scenarios, the residence time can be shortened by about half to yield a corresponding increase in mono-GTBE yield of about 50% (e.g., see FIG. 10 data where halving the time resulted in increasing yield from 20% to 30% mono-GTBE at 1:2 molar ratio and 65° C.). It can be seen that there is a peak zone of mono-GTBE yield at lower residence times, where the yield is above 25% (at 65° C.) and is above 20% (at 80° C.). Operating the production process within such a peak zone can thus yield higher concentrations of mono-GIBE (e.g., yields above 20%, 22%, 24%, 26%, 28%, or 30%).

### Experimentation 3

Experiments were conducted to assess to which extent pure mono-GIBE and pure di-GTBE could be converted to other GIBE compounds. The influence of temperature and reaction time on the reactor composition were investigated.

### Description of Setups

The experiments were performed in a high-pressure autoclave and a low-pressure glass setup for experiments in which there was no pressure build-up.

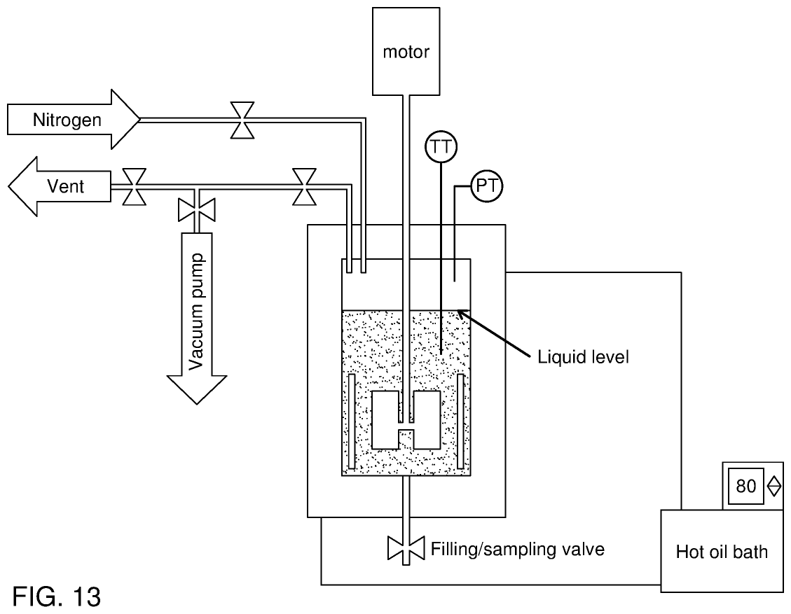

The 0.5 l high-pressure autoclave could handle pressures up to 200 bar and temperatures up to 250° C. The reactor was operated batchwise. A vacuum pump was used to evacuate air in the reactor. Nitrogen could be fed to the reactor. The reactor temperature was regulated with a Julabo heater. A blade stirrer was operated with a magnetic drive. Baffles inside the reactor promoted mixing. The temperature and pressure were monitored during each experiment. Sampling was performed via a sampling valve at the bottom of the reactor. Samples were analysed by gas chromatography. FIG. 13 shows a schematic representation of the experimental setup. Due to safety reasons, the stirrer motor and oil bath could not be left turned on overnight. Therefore, stirring and heating could only be performed during daytime.

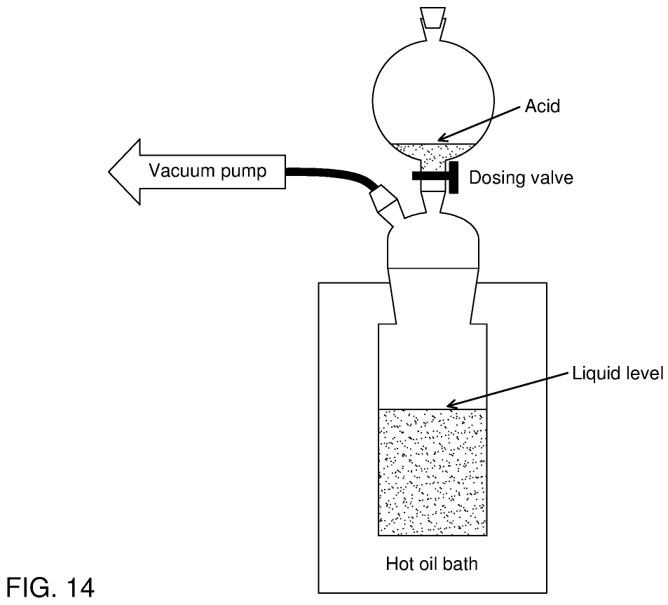

The low-pressure glass setup could be continuously operated for longer periods of time compared to the high-pressure autoclave with smaller amounts of GIBE (approximately 20 ml). The liquid was stirred with a magnetic stirrer. No intermediate sampling was performed, one sample after prolonged period of time provided the equilibrium composition. FIG. 14 shows a schematic representation of the experimental setup

### Results

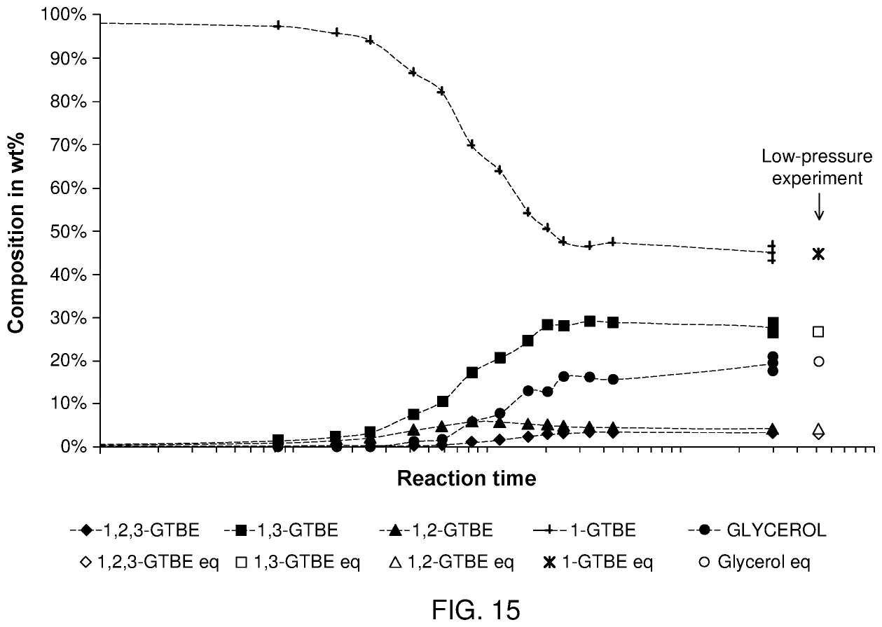

FIG. 15 shows the concentration profile obtained for the reaction of mono-GIBE with H₂SO₄ at a temperature of 50° C. (as an example of a pure GIBE with H₂SO₄ reaction). From FIG. 15 can be concluded that mono-GIBE is partially converted. The main components present at EQ are mono-GIBE, di-GIBE and glycerol. Both the high-pressure apparatus and the low-pressure apparatus resulted in the same composition at the end of reaction.

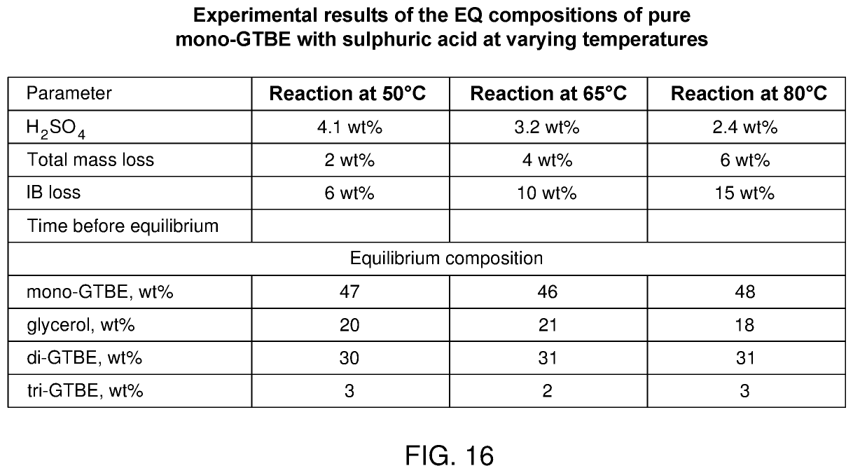

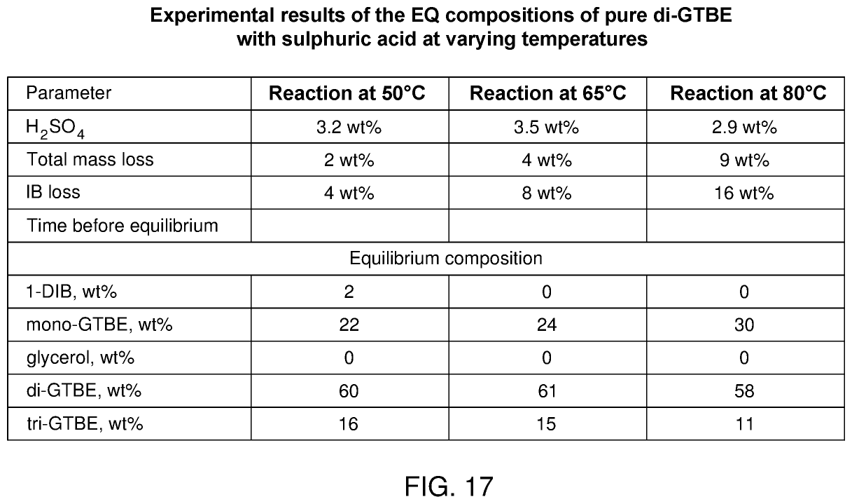

Tables 3 and 4, illustrated as FIGS. 16 and 17 respectively, show the equilibrium compositions of mono-GTBE with H₂SO₄ and di-GTBE with H₂SO₄ at varying temperatures respectively. From FIG. 16 (pure mono-GTBE) can be concluded that the temperature did not significantly affect the equilibrium composition. Only small amounts of tri-GTBE were formed. However, an increase in temperature resulted in an increased formation of isobutene. Unlike experiments performed with pure mono-GTBE, experiments performed with pure di-GTBE (FIG. 17) did not result in the formation of glycerol. Di-GTBE was partially converted. The main components at EQ are di-GTBE, mono-GTBE and tri-GTBE.

FIGS. 15 to 17 indicate that pure mono-GTBE and pure di-GTBE can be converted partially into other GTBE compounds.

### Experimentation 4

A tailor-made numerical program was previously developed to be able to perform thermodynamic model simulations of the production of GTBEs (ref: Wermink, W. N. et al.: “GTBE—Turning residual biodiesel glycerin into a remedy for diesel soot emissions; thermodynamic background,” 15^{th} European biomass conference and exhibition; from research to market development, 2007, p 2045-2049). A predictive function was implemented to be able to predict equilibrium compositions at varying settings, in particular varying temperatures and varying reactant compositions.

The thermodynamic model was constructed from equilibrium constant temperature relationships of the involved reactions in the production of GTBE from pure isobutene or pure tert-butyl alcohol and pure glycerol. The thermodynamic relationships were derived from experimental work and literature. The GTBE system is non-ideal, because the components present in the reacting system differ in polarity, structure and dimension respectively. The deviation from ideal behaviour was accounted for by introducing activity coefficients. Activity coefficients were calculated with an activity coefficient method based on group contribution theory. The predictive function is a numerical code with which the sets of mole fractions and activity coefficients at equilibrium are determined through iteration. It calculates the optimum solution for which the difference among the theoretical and calculated chemical equilibria of the different reactions occurring in the GIBE system is the smallest.

### Results

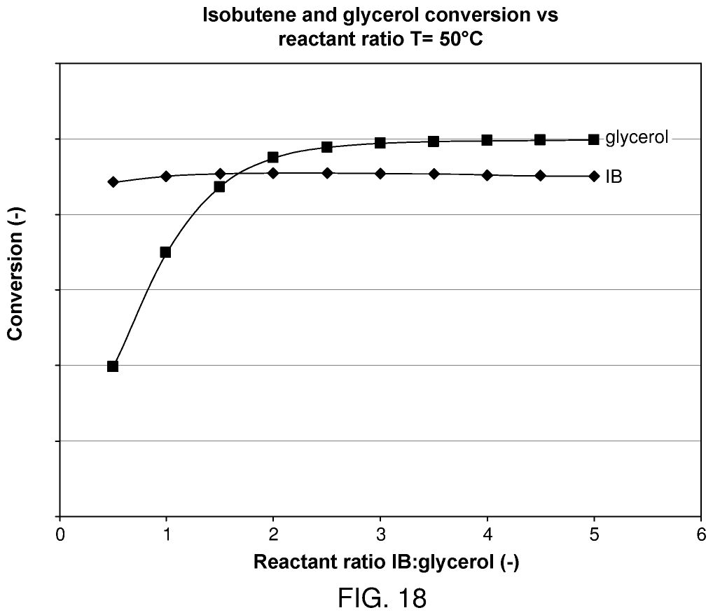

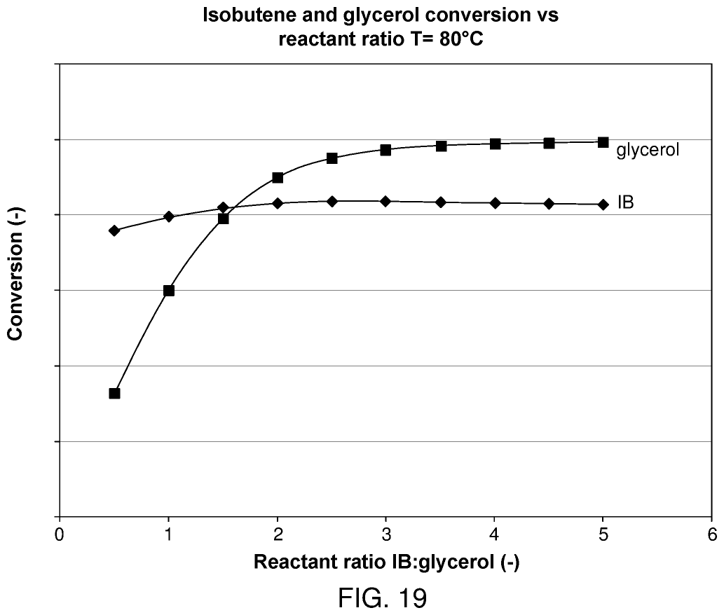

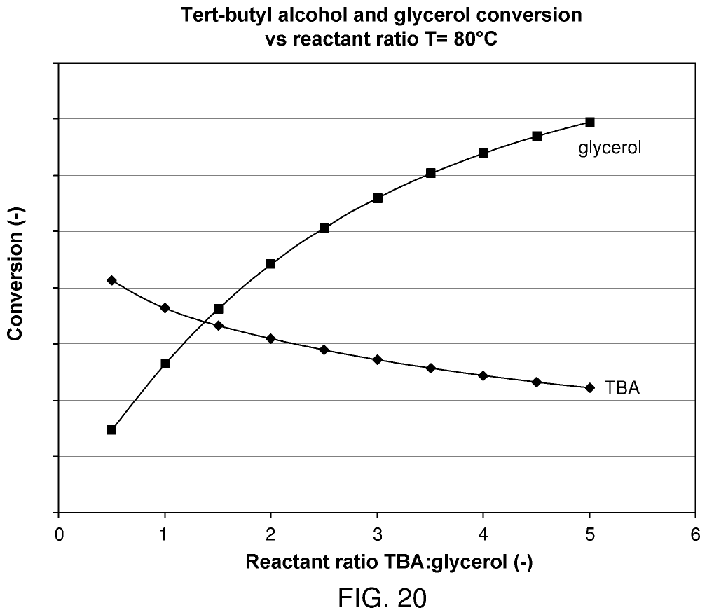

FIGS. 18 and 19 show the conversions of isobutene and glycerol as a function of the reactant ratio at temperatures of 50° C. and 80° C. respectively. FIG. 20 shows the conversions of TBA and glycerol as a function of the reactant ratio at a temperature of 80° C. An increase in temperature results in a slight decrease in both IB and glycerol conversions. Conversions are significantly lower when GIBE is produced from tert-butyl alcohol compared to isobutene.

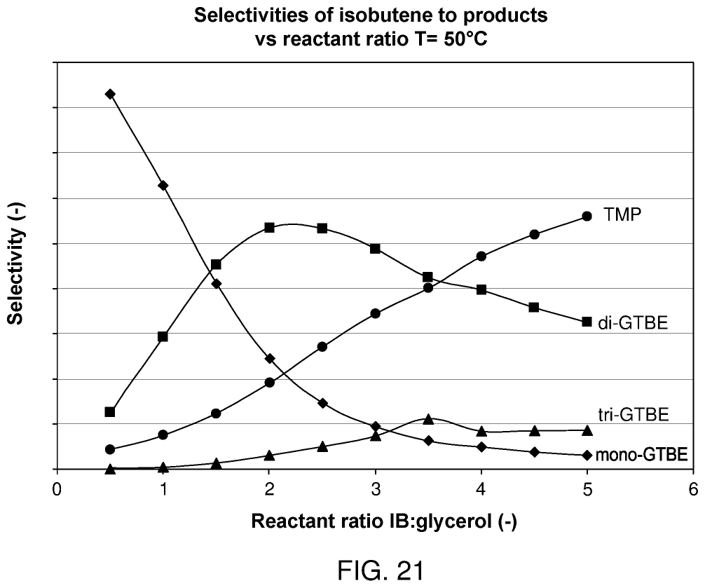

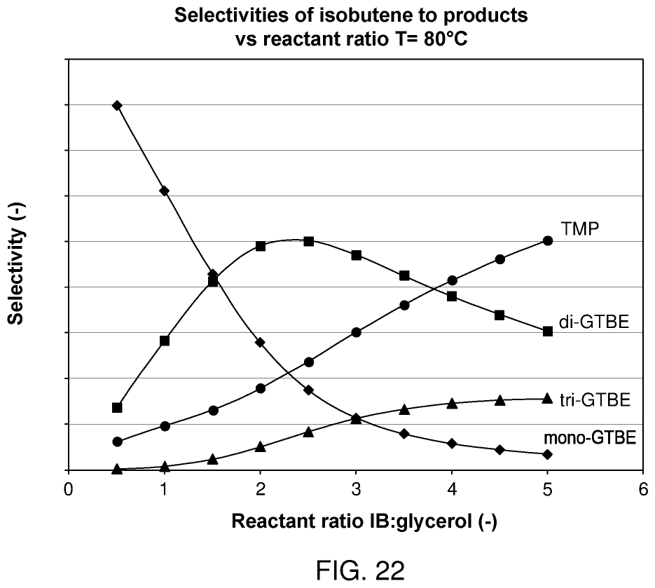

FIGS. 21 and 22 show selectivities of isobutene to products as a function of the reactant ratio at temperatures of 50° C. and 80° C. respectively. An increase in temperature slightly lowers the selectivity to di-GIBE and slighty increases the selectivity to tri-GIBE. An optimum in di-GIBE selectivity is observed at a reactant ratio of approximately v=2.2. Mono-GIBE has the highest selectivity till a reactant ratio of around 1.5, di-GIBE has the highest selectivity from a reactant ratio of around 1.5 to around 3.8 and for reactant ratios higher than 3.8 trimethylpentene has the highest selectivity.

From the results, it can be concluded that a change in reactant ratio significantly influences the equilibrium composition.

### Experimentation 5

Experiments were conducted to assess octane boosting effects of different GIBE types. To do so, a GIBE blend was separated into mono-GIBE, di-GIBE and tri-GIBE, and each was then subjected to octane and other tests. The separation included multiple vacuum distillation runs.

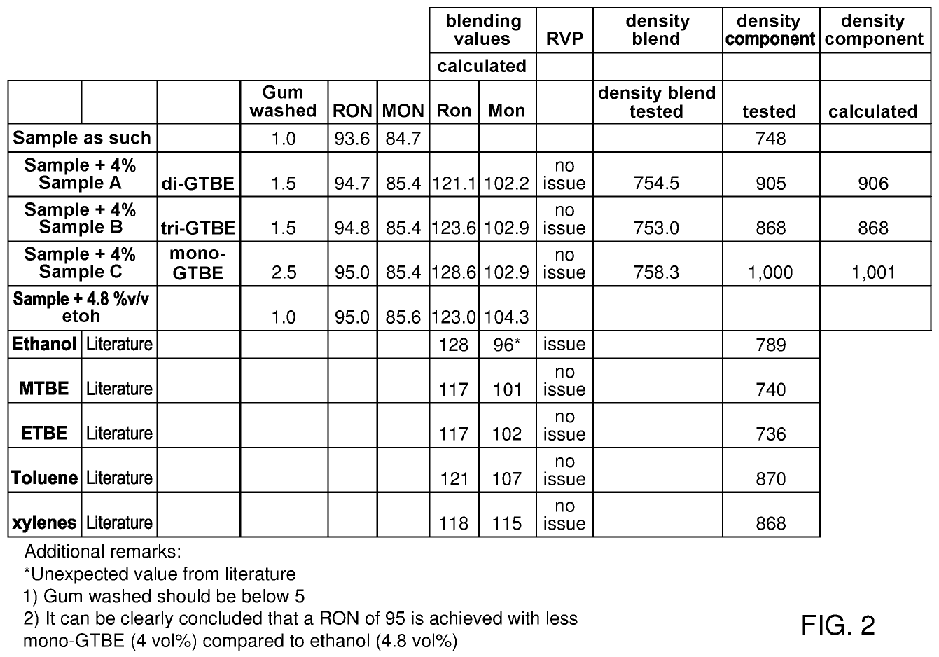

FIG. 2 presents results that illustrate effects of the different types of GIBE on the octane number. For example, it was found that mono-GIBE provides the highest octane boosting octane of all of the GTBEs that were tested, particularly the Research Octane Number (RON). The tests to determine density, RON, MON and Gum Content were conducted according to ISO 12185, EN ISO 5164, EN ISO 5163 and EN ISO 6246 respectively.

### Experimentation 6

Additional experiments to assess octane boosting of pure mono-GIBE and pure di-GTBE in fuels containing varying GIBE concentrations were conducted at facilities different from those of Experimentation 4. This investigation was performed a.o. to investigate whether an increase in GIBE content resulted in fuel mixing problems and to determine if an increase in GIBE content results in an increase in octane number measured.

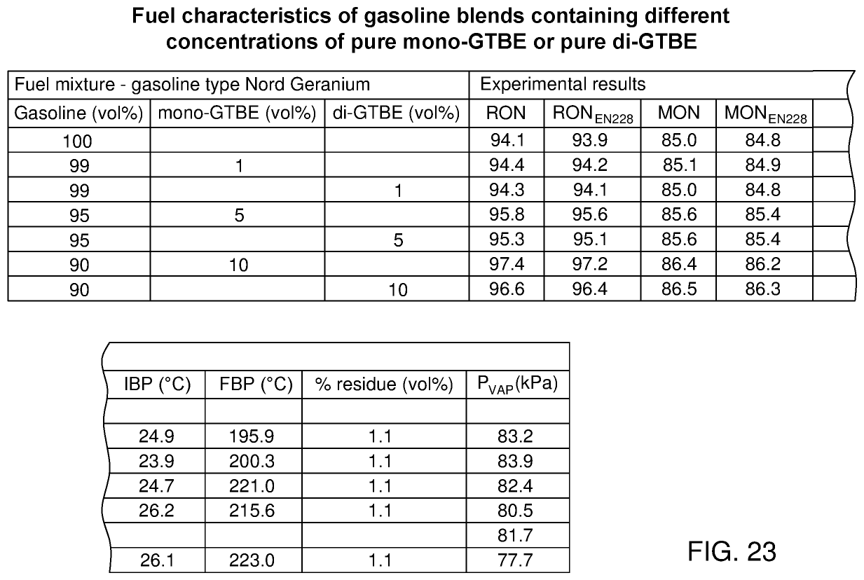

Table 5 illustrated as FIG. 23 shows the effects of pure mono-GIBE and pure di-GTBE, added in varying fractions to gasoline, on a.o. octane number and other fuel characteristics. For example, in agreement with Experimentation 5, it was found that mono-GIBE provides the highest boosting of octane of the GTBEs tested. Moreover, an increased content of both mono-GIBE or di-GIBE resulted in an increased boosting of octane. No fuel mixing problems were encountered with blending GTBEs up to a fraction of 10 vol %. An increase in final boiling point was observed with an increased content of pure mono-GIBE or pure di-GIBE. The tests to determine RON, MON, initial boiling point (IBP) and final boiling point (FBP), and vapour pressure (air saturated) were conducted according to the international standards IP 237, IP 236, IP 123 and IP 394 respectively.

### Experimentation 7

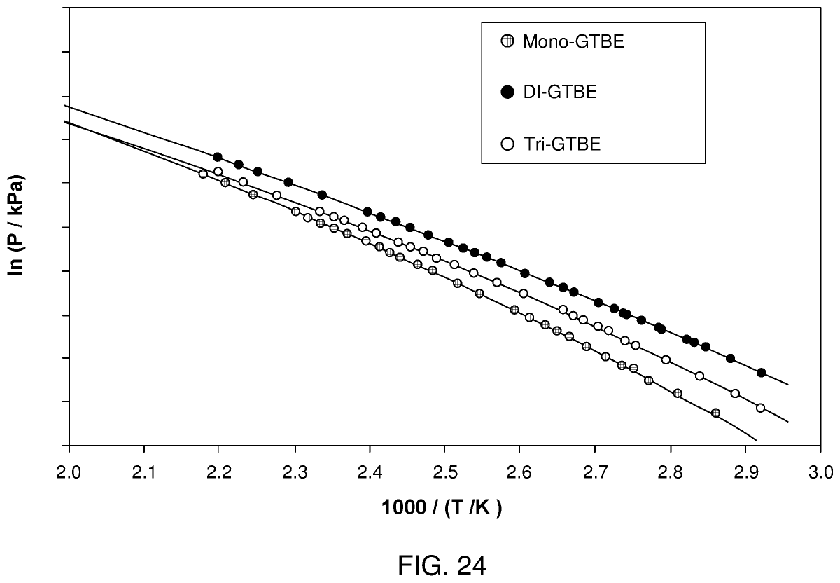

Experiments were performed to determine PT-curves of pure mono-GIBE, pure di-GTBE and pure tri-GIBE. The experimental data of the different GTBEs were fitted to the Clausius Clapeyron equation. FIG. 24 presents the PT curves of pure mono-GIBE, pure di-GIBE and pure tri-GIBE respectively. The dots represent the experimental points, the solid line represents the simulated points. For example, it was observed that the vapour pressure of di-GIBE is highest among the different GTBEs, and that the vapour pressure of mono-GIBE is lowest among the different GTBEs.

### Experimentation 8

As explained in Experimentations 1 to 4 a GIBE production run will always result in a mixture of GTBEs; pure GIBE compounds cannot be produced in a single reactor step. Further purification can be performed via for example vacuum distillation.

### Description of Setup

The vacuum distillation unit used for GIBE purification and separation was a batch-wise operated vacuum distillation unit consisting of an insulated glass column with a height of 1.5 m and packed with 5×5 mm Raschig rings. The setup is equipped with a temperature-controlled heater, a vacuum pump and a Julabo cooler to provide cooling liquid to the top condenser. Bottom and top temperatures, as well as pressure, were monitored. The sample in the bottom flask was stirred during distillation with a magnetic stirrer. A gas wash flask was connected between the vacuum pump and the top condenser to prevent liquids entering the vacuum pump.

### Results

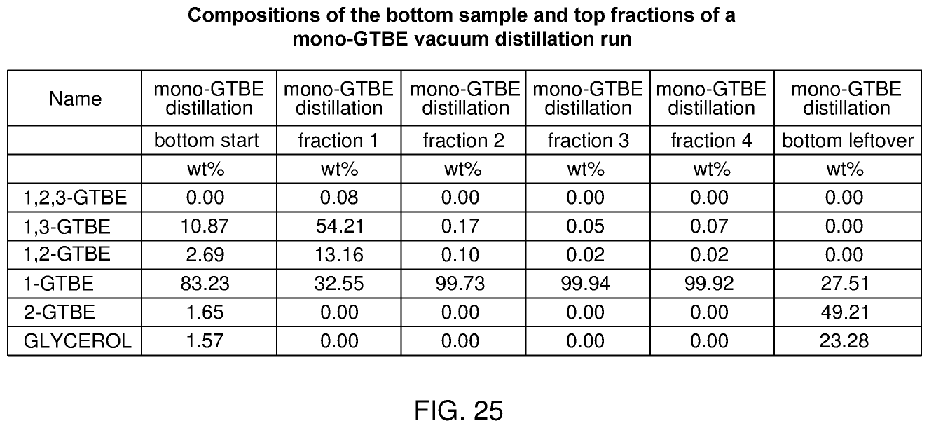

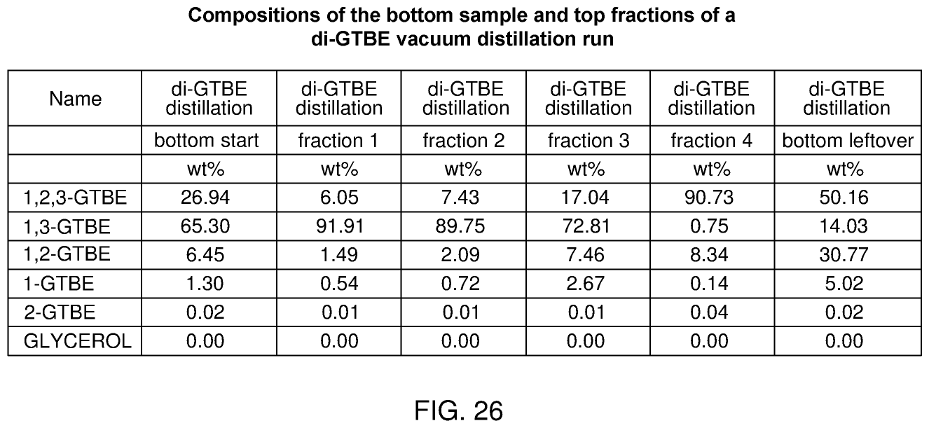

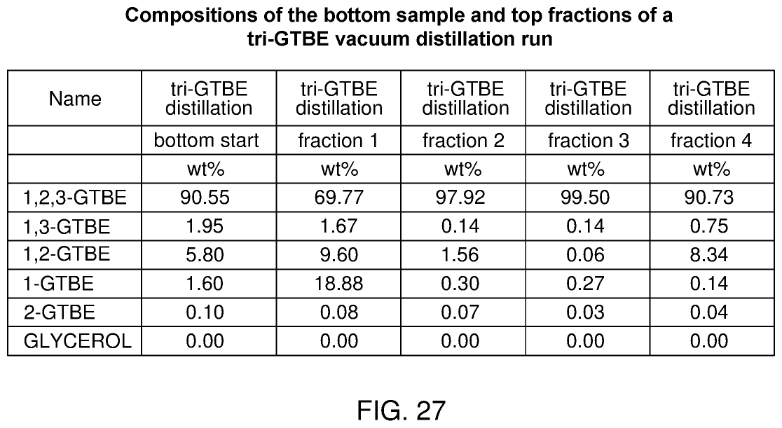

Table 6, illustrated as FIG. 25, presents the compositions of the bottom sample, containing a large amount of mono-GIBE, and the top fractions obtained during distillation. Table 7, illustrated as FIG. 26, presents the compositions of the bottom sample, containing a large amount of di-GIBE, and the top fractions obtained during distillation. Table 8, illustrated as FIG. 27, presents the compositions of the bottom sample, containing a large amount of mono-GIBE, and the top fractions obtained during distillation.

For example, it can be concluded that a mixture of GTBEs can be further purified to pure mono-GIBE, pure di-GIBE or pure tri-GIBE. Moreover, to obtain pure mono-GIBE, or pure di-GIBE, or pure tri-GIBE, several vacuum distillation runs could be required (depending on the amount of pure GIBE required, the composition of the GIBE mixture to be distilled and the vacuum distillation setup configuration and settings).

In addition, a commercially available GIBE-based blend sold as an octane booster was also tested for comparative purposes. This GIBE-based blend was tested and was found to include mono-GIBE, di-GIBE, tri-GIBE and a fair amount of trimethylpentene (TMP), with a mono-GIBE content of 4.6 wt %. The following table shows the breakdown of the composition according to GC analyses:

| | GC analysis 1 | GC analysis 2 |
|---|---|---|
| Compound | (wt %) | (wt %) |
| 1-TMP | 1.1 | 1.3 |
| 2-TMP | 19.5 | 20.8 |
| 1,2,3-GTBE | 0 | 0 |
| 1,3-GTBE | 19.2 | 17.0 |
| 1,2-GTBE | 53.5 | 54.2 |
| 1-GTBE | 4.6 | 4.6 |
| 2-GTBE | 0 | 0 |

### Experimentation 9

Experiments were performed to determine the densities and viscosities of pure mono-GTBE, pure di-GTBE and pure tri-GIBE as a function of temperature. FIG. 28 presents the densities of pure mono-GIBE, pure di-GTBE and pure tri-GIBE respectively as a function of temperature. For example, it was observed that the density of mono-GIBE is highest among the different GTBEs, and that the density of tri-GIBE is lowest among the different GTBEs. FIG. 29 presents the dynamic viscosities of pure mono-GIBE, pure di-GIBE and pure tri-GIBE respectively as a function of temperature. For example, it was observed that the dynamic viscosity of mono-GIBE is highest among the different GTBEs, and that the dynamic viscosity of tri-GIBE is lowest among the different GTBEs.

1. A method of boosting octane of gasoline, comprising:
contacting glycerol and isobutene in a reaction vessel under conditions to generate a blend of Glycerol Tert-Butyl Ethers (GTBE) comprising mono-GTBEs, di-GTBEs and tri-GTBE;
regulating the conditions to favor production of mono-GTBEs according to equilibrium reactions, thereby producing a mono-shifted GTBE blend;
using the mono-shifted GTBE blend with gasoline as an octane booster.

2.-109. (canceled)
